(19) United States
(12) Patent Application Publication
(10) Pub. No.: US 2017/0130196 A1
(43) Pub. Date: May 11, 2017

(54) METHOD FOR CULTURING AND PATTERNING CELLS

(71) Applicant: President and Fellows of Harvard College, Cambridge, MA (US)

(72) Inventors: Gulden CAMCI-UNAL, Cambridge, MA (US); George M. WHITESIDES, Newton, MA (US)

(21) Appl. No.: 15/346,305

(22) Filed: Nov. 8, 2016

Related U.S. Application Data
(60) Provisional application No. 62/252,661, filed on Nov. 9, 2015.

(51) Int. Cl. C12N 5/00; C12N 1/20; C12Q 1/04

(52) U.S. Cl. CPC C12N 5/0068; C12Q 1/04; C12N 1/20; C12N 2533/54; C12N 2533/50; C12N 2535/10; C12N 2513/00

(57) ABSTRACT

A cell scaffold includes a string having a diameter of less than 500 μm; a hydrogel matrix supported on the string; and cells seeded onto the hydrogel matrix. The scaffold is used to cultivate cells, and to evaluate cell viability or cell metabolic activity.

### RELATED APPLICATIONS

This application claims the benefit of priority under 35 U.S.C. §119(e) to co-pending Application No. 62/252,661, filed Nov. 9, 2015, the contents of which are incorporated by reference in their entirety.

### INCORPORATION BY REFERENCE

All patents, patent applications and publications cited herein are hereby incorporated by reference in their entirety in order to more fully describe the state of the art as known to those skilled therein as of the date of the invention described herein.

### BACKGROUND

Three-dimensional (3D) structures of hydrogels such as collagen or Matrigel are useful supports for cells but have several disadvantages, such as, i) they are mechanically weak, and very difficult to form and manipulate with small (e.g., <mm) dimensions; and ii) unless dimensions are small, rates of mass transport of O₂, glucose, and waste products make it impossible for cells supported in them to maintain usual metabolism. Those disadvantages notwithstanding, hydrogels have been explored extensively as matrices for cell growth. They are, in some limited ways, demonstrate similarities to the extracellular matrix (ECM) of tissues, and can be used for delivery of cells, screening of therapeutics for their influence on cells, and studying vascularization. 3D hydrogel scaffolds mimic some aspects of the microenvironments of tissues, and are useful in studying cellular alignment, differentiation, and response to toxins. Hydrogels also make it possible to control spatial distribution of cells in 3D.

Hydrogels with dimensions smaller than 200 μm do not limit the rate of diffusional transport of nutrients and waste to/from cells, and therefore can be used to encapsulate cells without compromising metabolism. However their mechanical fragility makes them difficult to manipulate. While microfabrication can generate reinforced hydrogels, the requirement for multi-step procedures, expensive consumables, and specially designed devices make microfabrication inconvenient for cell biologists. There is, thus, a need for methods to handle, culture, and pattern cells that combine the advantages of hydrogels as matrices with more convenient structures than hydrogels that combine mechanical stability, ease of fabrication, and dimensions that circumvent problems of nutrient deficiency (especially anoxia) that come from metabolism and diffusional mass transport.

### SUMMARY

In one aspect, a simple method for culturing and patterning cells in 3D is provided. In particular embodiments, off-the-shelf, commercially available strings are provided as in vitro cell culture platforms that can be used to generate biomimetic structures by patterning cells. In one or more embodiments, strings with different diameters (150-200 μm) can be used as supporting materials to culture cell-laden hydrogels. The cells-in-string (CIS) system can easily be shaped or manipulated to yield in various geometrical patterns. Because the diameter of the strings is small (<200 μm), cells do not suffer from mass-transport limitations to the availability of O₂ and nutrients.

In another aspect, tissue-like structures that include a cells-in-string system sandwiched between two layers of hydrogel-impregnated paper are also provided. Cell-containing string can be used as a template to pattern gel and gel-supported cells on paper. The cells-in-string system supports high cell viability, maintains metabolic activity of cells, improves alignment and elongation of cells, can be used to generate patterns of cells with 150-200 μm dimensions, allows for patterning cells on paper, and enables co-culturing different types of cells. Cells-in-string systems also allow testing of the effects of toxic chemicals on cells.

The cells-in-string approach enables generation of new and simple in vitro systems for cell cultures. Cells-in-string is a simple tool that can be adapted by any research facility without the need for sophisticated instrumentation.

In one aspect, a cell scaffold includes a string having a diameter of less than 500 μm; and a hydrogel matrix supported on the string; and cells seeded onto the hydrogel matrix.

In one or more embodiments, the cells are bacterial cells, insect cells, yeast cells, or mammalian cells.

In one or more embodiments the hydrogel is a temperature sensitive hydrogel, the hydrogel is an ionotropic hydrogel, and for example, the hydrogel is selected from alginic acid (AA), carboxymethylcellulose (CMC), [symbol] -carrageenan, poly(galacturonic acid) (PG), poly(bis(4-carboxyphenoxy)-phosphazene, guar gum, gellan gum, PuraMatrix hydrogel, poly(ethylene glycol) (PEG), poly(2-hydroxyethyl methacrylate) (PHEMA), poly(N-isopropylacrylamide) (PNIPAAM) gelatin, heparin, agarose, fibrin, pullulan, and dextran.

In one or more embodiments, the hydrogel is collagen or Matrigel hydrogel.

In any of the preceding embodiments, the string has a diameter in the range of 50-500 μm, or 100-400 μm, or 100-200 μm.

In another aspect, a sheet structure is provided including at least one cell scaffold as described herein; and a pair of hydrogel impregnated sheets, wherein the at least one cell scaffold is disposed between the pair of hydrogel sheets.

In one or more embodiments, sheet structure includes first and second cell scaffolds.

In any preceding embodiment, the first cell scaffold includes cells of a first type and the second cell scaffold includes cells of a second type.

In any preceding embodiment, the first and second cell scaffold include cells of a first cell type.

In one or more embodiments, the at least one cell scaffold includes cells of a first cell type; and further include a second string comprising an agent of interest, wherein the at least one cell scaffold and the second string are disposed between the pair of hydrogel sheets and in contact with one another.

In any preceding embodiment, the agent of interest is a toxin, drug, serum, growth factor, cytokine, neurotransmitter, chemoattractant, or anti-mitotic agent.

In any preceding embodiment In any preceding embodiment, the sheets are paper.

In any preceding embodiment, the paper is a cellulosic paper.

In another aspect, a method of culturing cells includes providing a cell scaffold as described herein; and incubating the cell scaffold under conditions that promote cell growth.

In another aspect, a method of evaluating cell viability includes providing a cell scaffold as described herein; incubating the cell scaffold; and qualitatively or quantitatively determine the level of viable cells in the cell scaffold.

In another aspect, a method of evaluating cell metabolic activity includes providing a cell scaffold as described herein; and incubating the cell scaffold in the presence of an agent capable of assessing metabolic activity; and qualitatively or quantitatively determining the level of metabolic activity as a function of the agent capable of assessing metabolic activity.

In another aspect, a method of patterning cells onto surface includes providing a first cell scaffold as described herein having a first cell type; disposing the first cell scaffold between a pair of hydrogel sheets; incubating the cell scaffold-hydrogel sheet assembly; and separating the incubated cell scaffold from the pair of hydrogel sheets, wherein the first cell type is patterned on the pair of hydrogel sheets.

In one embodiment, the method of patterning cells onto surface further includes providing a second cell scaffold as described herein having a second cell type; disposing the second cell scaffold between a pair of hydrogel sheets; incubating the cell scaffold-hydrogel sheet assembly comprising the first and second cell scaffolds; and separating the incubated cell scaffold from the pair of hydrogel sheets, wherein the first cell type and the second cell type are patterned on the pair of hydrogel sheets.

### BRIEF DESCRIPTION OF THE DRAWING

The invention is described with reference to the following figures, which are presented for the purpose of illustration only and are not intended to be limiting.

In the Drawings:

FIG. 1 is a schematic illustration of the preparation and use of a cells-in-string scaffold according to one or more embodiments.

FIG. 2 illustrates a tissue-structure according to one or more embodiments including one or more cells-in-string scaffolds disposed between gel impregnated paper sheets.

FIG. 3A are fluorescent confocal microscopy images of threads in a collagen matric (i) or a Matrigel matrix (ii) loaded with NIH-3T3 fibroblast cells stained with Calium AM indicating live cells and ethidium homodimer indicating dead cells; cell-loaded cotton threads are shown in image on left and cell-loaded silk threads are shown in image on left.

FIG. 3B are fluorescent confocal microscopy images of threads in a collagen matric (i) or a Matrigel matrix (ii) loaded with A549 human lung cancer cells stained with Calium AM indicating live cells and ethidium homodimer indicating dead cells; cell-loaded cotton threads are shown in image on left and cell-loaded silk threads are shown in image on left.

FIG. 3C is a bar graph of % viability for NIH-3T3 fibroblast cells and A549 cells on silk and cotton threads in collagen and Matrigel. The results show that the viability of cells within the cell scaffolds under all conditions was greater than 89% (n=3).

FIG. 4A are fluorescent confocal microscopy images of the NIH-3T3 cells stained with F-actin and DAPI in (i) Matrigel or (ii) Collagen (right) in silk string with fluorescent confocal microscope following 14 days in culture.

FIG. 4B are fluorescent confocal microscopy images of A549 cancer cells stained with F-actin and DAPI in (i) Matrigel or (ii) Collagen (right) in silk strings after 14 days in culture.

FIGS. 5A and 5B are bar graphs plots of fluorescent intensity over time illustrating the metabolic activity of the NIH-3T3 fibroblast cells in Matrigel and Collagen matrices in cotton and silk, respectively.

FIGS. 5C and 5D are bar graphs plots of fluorescent intensity over time illustrating the metabolic activity of the A549 lung cancer cells in Matrigel and Collagen matrices in cotton and silk, respectively.

FIG. 6A illustrates patterning for cell-laden string constructs in 3D in one or more embodiment, in which the NIH-3T3 fibroblast (60), A549 lung-cancer (62), T84 colon carcinaoma (64), and H9 lymphocyte (66) cells were patterned in single, cross, and triangular shapes in cotton strings for two days. Scale bars represent 150 μm.

FIG. 6B illustrates patterning for cell-laden string constructs in 3D in one or more embodiment, in which cell-laden strings (NIH-3T3 fibroblast (61), A549 (63), D1.1 T-lymphoblast (65) H9 lymphocyte (67) cells in cotton strings, and then sandwiched either single or multiple strings between two layers of filter paper impregnated with Matrigel or cells-in-Matrigel) as a micro-patterning tool. Paper was imaged after two days in culture with confocal microscopy. Scale bars represent 150 μm.

FIG. 6C illustrates patterning for cell-laden string constructs in 3D in one or more embodiment, in which GFP-A549 (68) cells in cotton string on a collagen gel to generate patterns of cells. Scale bars represent 150 μm.

FIGS. 7A and 7B illustrate the use of cells-in-string as an in vitro drug testing platform in one or more embodiments, in which FIG. 7A shows green fluorescent protein (GFP)-expressing A549 human lung cancer cells grown in silk string for two days and used as a control; and

FIG. 7B shows green fluorescent protein (GFP)-expressing A549 human lung cancer cells grown in silk string for two days to which 1 μM Doxorubicin was embedded in the top string, which was removed following 24 h culture period. GFP-A549 cells died in the area where they were exposed to the top string. Scale bar represents 150 μm.

FIG. 8 demonstrates the growth of bacteria on string stained with sytox green (indicating live cells) and propidium iodide (indicating dead cells) according to one or more embodiments.

### DETAILED DESCRIPTION

Hydrogels that are thinner than 200 μm are challenging to handle and manipulate for generation of three-dimensional (3D) cellular constructs. In one aspect, the invention provides a simple platform for culturing and patterning cells in 3D. Simple, low-cost, and widely commercially available strings are used as in vitro cell culture platforms and to generate biomimetic structures by patterning cells. In some embodiments, the cells are bacterial cells, insect cells, yeast cells, fungi, plant cells, or mammalian cells. In preferred embodiments, the cells are mammalian cells.

The cells-in-string system supports high cell viability, maintains high metabolic activity of cells, improves alignment and elongation of cells, can be used to generate patterns in μm-mm scale, allows for patterning cells on paper, and enables co-culturing different types of cells. Cells-in-string can also be used to test the effects of toxic chemicals on cells.

In one or more embodiments, the string can be any commercially available string that is capable of sterilization. The string can be made of natural materials, such as silk or cotton, or synthetic polymers, for example, biodegradable polymers. Exemplary natural and synthetic commercial strings include cotton, silk, nylon, polyester, linen, wool, metallic, rayon, bamboo, flax, jute, and poly(lactic-co-glycolic acid) (PLGA) strings. The strings can have a range of thicknesses; however, preferable string diameter is less than 500 μm, or less than 400 μm, or less than 300 μm or less than 200 μm (or any range bounded by any value stated herein) so that the cell in string scaffold is not diffusion-limited.

In one or more embodiments, the strings are used as 3D scaffolds to culture different types of cells in the presence of a hydrogel matrix. Hydrogel matrices have been widely used in cell culturing and any hydrogel suitable for use in cell culturing can be used. Exemplary hydrogel matrices include collagen and Matrigel hydrogel. In one embodiment, the hydrogel is a temperature sensitive hydrogel. In particular embodiments, the temperature-sensitive hydrogel is Matrigel or collagen. In some embodiments, the hydrogel is an ionotropic hydrogel. In particular embodiments, the ionotropic hydrogel comprises alginic acid (AA), carboxymethylcellulose (CMC), [symbol] -carrageenan, poly(galacturonic acid) (PG), poly(bis(4-carboxyphenoxy)-phosphazene, guar gum, gellan gum, or PuraMatrix. In some embodiments, the hydrogel comprises poly(ethylene glycol) (PEG), poly(2-hydroxyethyl methacrylate) (PHEMA), poly(N-isopropylacrylamide) (PNIPAAM) gelatin, hyaluronic acid, heparin, agarose, fibrin, pullulan, and dextran. One or more hydrogels can be used for cell culturing. The cultured cells can be used in a variety of tests or assays to evaluate cell properties or behaviour.

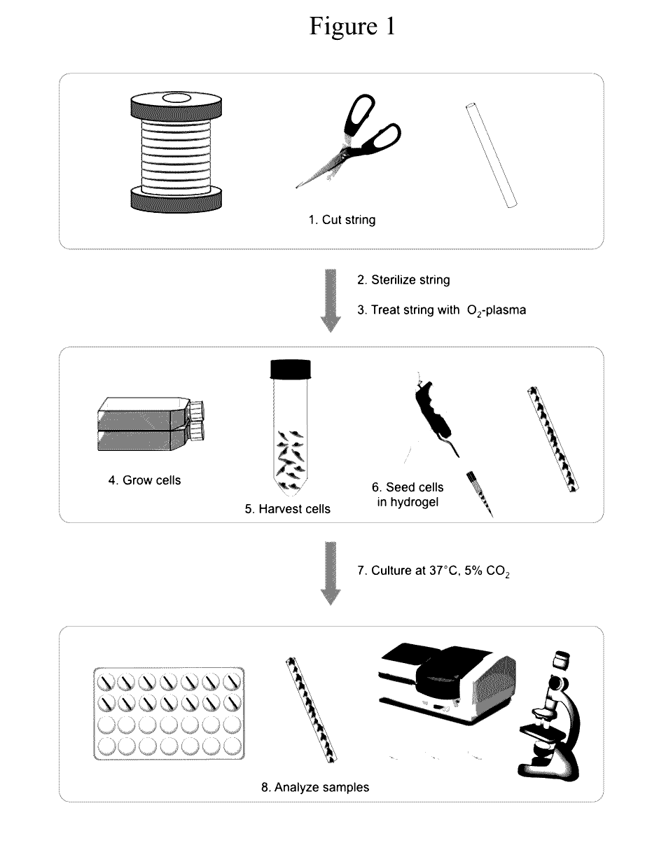

FIG. 1 illustrates an exemplary preparation and use of a string scaffold for culturing cells. In an initial step (1), commercially available cotton, or silk strings can be cut to any desired length (1 mm-100 mm). The particular string length can be selected dependent of the intended culturing application. For instance, strings with 5 mm, 10 mm, or 30 mm length can be used in 96-well plates, 24-well plates, and 6-well plates, respectively; whereas strings with 100 mm length can be utilized for culturing cells in petri dishes. Strings with different diameters, e.g., ranging from 100 to 500 μm, e.g., 100 to 200 μm, can be used as supporting materials to culture cell-laden hydrogels. Strings possess fibrous network that can support mechanically weak hydrogels. Cells-in-string (CIS) constructs can easily be shaped or manipulated to yield in various geometrical patterns. Due to the small diameter of the strings, in some embodiments the resulting cells-in-string scaffolds are not diffusion-limited.

Next, the string is sterilized as is shown in FIG. 1, step (2). Sterilization can be accomplished using any standard technique, such as by heat (autoclave, steam, or dry heat), chemical sterilization (ethylene oxide, nitrogen dioxide, ozone, glutaraldehyde and formaldehyde or hydrogen peroxide) or ionizing or non-ionizing radiation. The string is then optionally treated with O₂-plasma to render it hydrophilic as shown in FIG. 1, step (3). O₂-plasma can be used to change the hydrophilic properties of the string, modifying its compatibility with the hydrogel. For example, treatment can improve surface adhesivity of the scaffold for optimal seeding. Next, cells are grown and then harvested as shown in FIG. 1, step (4), using standard techniques appropriate for the cells of interest. The harvested cells are suspended in a matrix, such as Collagen type I or Matrigel, shown in FIG. 1 in step (5), and seeded on the strings as shown in FIG. 1, step (6). The seeding can be accomplished using known methods, such as depositing, e.g., by pipetting the cell/hydrogel suspension onto the strings or spraying the gel suspension onto the string. Alternatively, the string can be immersed in the cell/hydrogel suspension. While this specific example shows the cells and gel being combined first, before being applied to the string, it is also possible to apply the two to the string in separate steps. The cell-laden strings are cultured, for example, at 37° C. in a 5% CO₂-supplemented incubator, FIG. 1, step (7), however, any cell culturing process and culturing medium suitable for the cells of interest can be used. After the appropriate time, the cultured can then be analysed, FIG. 1, step (8), with different analytical techniques for viability, metabolic activity, cytoskeletal arrangement, and expression of proteins by the cells (by way of example). The cell scaffolds are robust and provide cell viability for day, weeks and even months. In some embodiments, the cells-in-string (CIS) constructs can be used for short-term (7 days or less) and long-term (2 to 12 weeks) cultures.

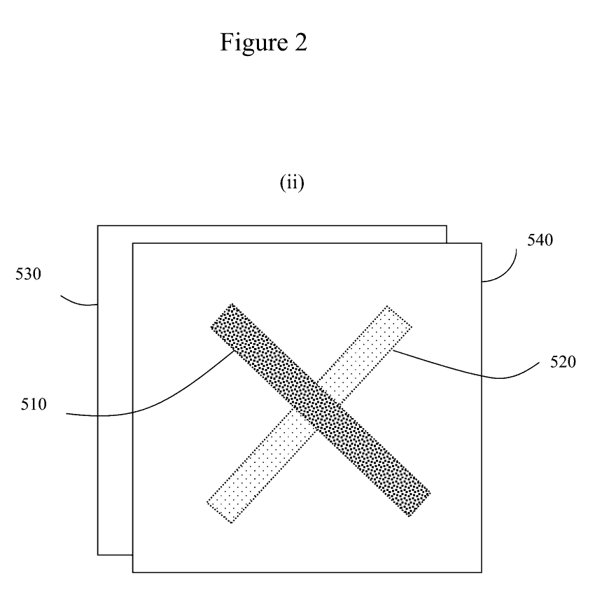

In another aspect, tissue-like structures can be prepared that include cells-in-string scaffolds sandwiched between two layers of hydrogel-impregnated paper. FIG. 2 illustrates an exemplary tissue-like structures including a pair of cells-in-string scaffolds 510, 520, sandwiched between hydrogel-impregnated papers 530, 540, according to one or more embodiments. As illustrated in FIG. 2, the cells-in-string scaffolds optionally contact each other as well as the paper sheets. The paper sheets can be any fibrous sheet and in some embodiments can be cellulosic sheets. In one or more embodiments, the paper is highly porous and capable of high loading of hydrogel. An exemplary paper is filter paper. Optionally the strings can be loaded with the same or different components. For example, the strings can be impregnated with the same or different cells. In other embodiments, one string can be impregnated with cells and the other can be loaded with a cell-modulating agent, such as a protein, peptide, serum, growth factor, or cytokine.

In one embodiment, a system and method are provided for the culturing of cells on a string scaffold. The string scaffold includes a string, fiber or thread having a diameter of less than 500 μm, or less than 400 μm, or less than 300 μm or less than 200 μm (or any range bounded by the values stated herein), so that the cell in string scaffold is not diffusion-limited. The string scaffold also includes a hydrogel matrix that is supported by the string scaffold. Cells to be cultured are seeded onto the hydrogel and string. In one embodiment, the cells can first be introduced into the hydrogel and the cell/hydrogel mixture is applied to the string scaffold. In other embodiments, the hydrogel is applied first to the string scaffold and harvested cells are applied in a subsequent step. In some embodiments, the gel is applied in a low viscosity state to the string scaffold to encourage wicking and penetration. Once applied, the gel composition is treated to initiate gelling, using known methods such temperature for temperature sensitive hydrogels or ionic concentrations for ionotropic hydrogels. The cells are cultured, for example, at 37° C. in a 5% CO₂-supplemented tissue incubator or other suitable incubator.

In one embodiment, a system and method are provided for evaluating the cellular organization of cells in the cells-in string scaffold. Cells are seeded and cultured as described herein above. The cells can be removed from the incubator, stained and examined, for example, by fluorescent confocal microscopy, to determine the organization of the cellular structure within the string. Organization of cells can provide insight about distribution and alignment of cells as well as demonstrating whether the cells formed proper connections among each other.

In one embodiment, a system and method are provided for evaluating the metabolic activity of cells. Cells are seeded and cultured as described herein above, except that the cells are incubated with an agent capable of assessing metabolic activity. For example, the cells on string constructs can be incubated with Alamar Blue, an agent that changes from blue to red in the presence of metabolically active cells. Other metabolic reagents may include MTS, MTT, XTT, and WST-1 assays.

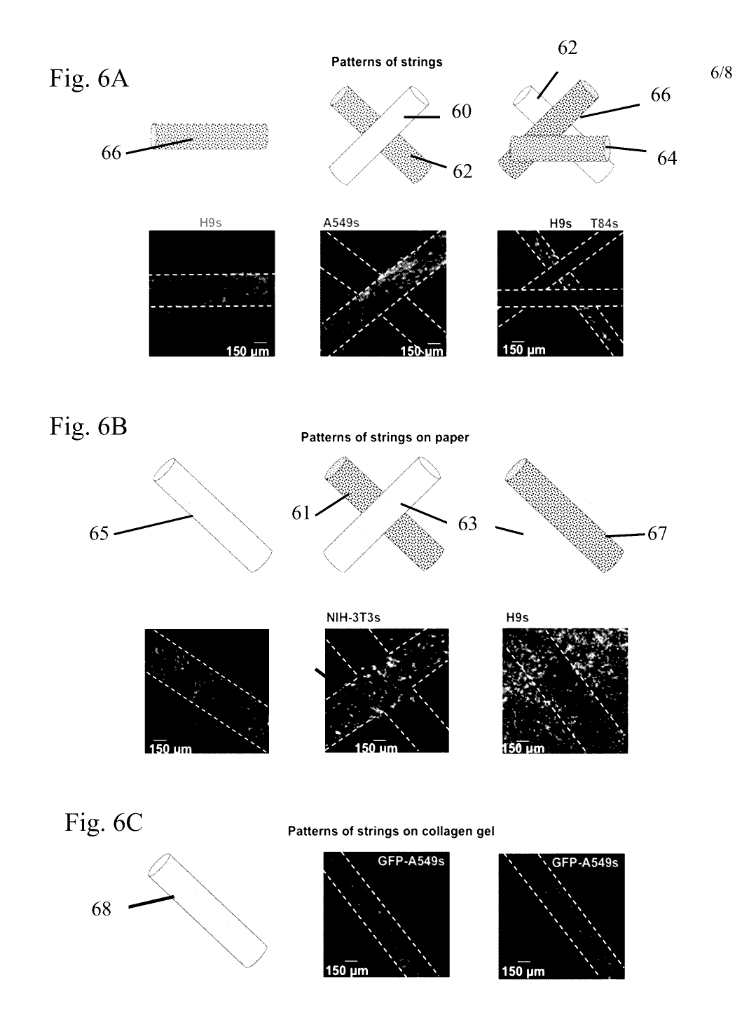

In one embodiment, a system and method are provided to pattern cells on paper scaffolds. Cells are seeded onto a plurality of string scaffolds as described herein above. The cells in strings can be the same or different. The strings are sandwiched between hydrogel impregnated paper sheets and incubated, whereupon the cells proliferate and are transferred onto the paper sheet. See, e.g., FIG. 6B. The hydrogel-impregnated paper sheets can include the same hydrogels uses for the seeding of cells on strings (as described herein above). Alternatively, the hydrogel matrices for the string and the paper can be different. On separation of the string scaffold from the paper sheet, patterned cells remain on the hydrogel impregnated paper sheet. Template-guided growth of cells in 3D was assessed by confocal microscopy. In one or more embodiments, the spatial distribution of cells is controlled by patterning them on paper. This system can be considered as a model for generation of co-cultures of different types of cells. For example, a vascularized muscle construct can be fabricated using endothelial cells in the string and muscle cells in the paper scaffolds. The cells in string scaffold and be used to generate vascular patterns in engineered scaffolds.

In one or more embodiments, a method and system for evaluating cell response to an agent of interest is provided. Cells are seeded onto a string scaffold as described herein above. A second string is embedded with an agent of interest. For example, the agent of interest can be a toxin, drug, serum, growth factor, cytokine, neurotransmitter, chemoattractant, or anti-mitotic agent. The string scaffolds containing the cells and the agent of interest are placed in contact with one another (e.g., they overlap one another) and are sandwiched between hydrogel impregnated paper sheets and incubated. The cells provide response to the target of interest, for example, by demonstrating increased metabolic activity, greater cell proliferation, reduced cell viability and the like, which can be observed and quantified.

In another embodiment, a three-dimensional cells-in-string system is provided for the identification and/or evaluation of cell activity, function or behaviour, including but not limited to alignment (e.g. muscle), differentiation (e.g. cardiac), and response to toxic chemicals (e.g. metals ions, drugs, therapeutics) or co-cultures (e.g. cancer cells, immune cells, fibroblasts). In other embodiments, the cellular function is proliferation, migration, viability, or gene transcription. The platform can include one or more cells impregnated with a gel matrix; the gel matrix can include cells that are the target of identification or evaluation. The platform can further include a well plate sized to accommodate a cells-in-string scaffold in the wells.

In one or more embodiments, cells-in-string are used to make vascularized structures by using endothelial cells-in-string in combination with 3D multi-layer paper scaffolds. The string scaffolds can also be used to mechanically or electrically stimulate the cells. The ability to provide external stimuli to cells helps controlling growth, alignment, and differentiation. The stacking process can be performed as given in FIG. 6B to generate vascularized microstructures using strings. Alignment of cells through micropatterning may potentially help them to differentiate into different lineages and control cell function.

Cells in string constructs provide at least four advantages: i) it uses commercially available strings, which provides an inexpensive way to fabricate 3D constructs; ii) it provides uniform distribution of cells within the scaffold, iii) it can be used for long-term cell cultures, and iv) it enables co-culture of different types of cells. The cells-in-string scaffold supports high cell viability, maintains cellular metabolic activity of cells, improves alignment and elongation of cells, can be used to generate patterns in a micrometer-to-mm scale, allows for patterning cells on paper, and enables co-culture of different types of cells.

The invention is illustrated with reference to the following examples, which are presented for the purpose of illustration only and are not intended to be limiting of the invention. The simple platform made up of strings and paper for culturing and patterning cells is described. The examples demonstrate that one can use commercially available strings that are impregnated with cells in a hydrogel matrix as in vitro cell culture platforms. The cells-in-string platform can also be used to form patterns of cells in 3D.

### Cell Cultures

NIH-3T3 cells were grown in DMEM medium supplemented with 10% FBS and 1% Pen-Strep. Upon reaching 70-80% confluence, the cells were passaged into new flasks. Similarly human lung-cancer cells, A549s, were cultured in DMEM medium supplemented with 10% FBS and 1% Pen-Strep. Human lymphoma cells, H9 and D1.1, were cultured in RPMI medium supplemented with 10% FBS and 1% Pen-Strep.

### Preparation of String Scaffolds

Cotton and silk strings were cut to 1 cm length and sterilized them by autoclaving. The strings were then by oxygen plasma for 30 sec, subsequently suspended the cells in collagen type I (2.5 mg/mL) or Matrigel at a concentration of 10×10⁶ cells/mL and then seeded on the plasma-treated string scaffolds. The cell-laden strings were placed in warm media to facilitate gelation at 37° C. and then the constructs were cultured in a 5% CO2-supplemented tissue incubator.

### Evaluation of Cell Viability in Cells-in-String

The strings were evaluated for cytotoxic effects for the mammalian cells in the cells in string system. The constructs were cultured in a 5% CO₂-supplemented tissue incubator for two weeks in the case of the NIH-3T3 fibroblasts and A549 human lung cancer cells in cotton and silk strings. The cytotoxicity for the mammalian cell lines in the cells in string construct were evaluated using a Live/Dead assay by incubating the cell-laden string constructs with 4 mM Calcein AM and 2 mM ethidium homodimer in DPBS for 30 min. The samples were rinsed with DPBS three times and subsequently imaged by a Zeiss LSM710 confocal microscope (Carl Zeiss Microscopy, LLC, Thornwood, N.Y.). Calcein-AM penetrates the membrane of live cells and is hydrolyzed to green fluorescent Calcein by the cellular esterases that are present in the live cells. Ethidium homodimer passes through the compromised membrane of the dead cells intercalating with DNA and forming red fluorescent color. Calcein AM indicates live cells (green) whereas ethidium homodimer shows the dead cells (red) in the images.

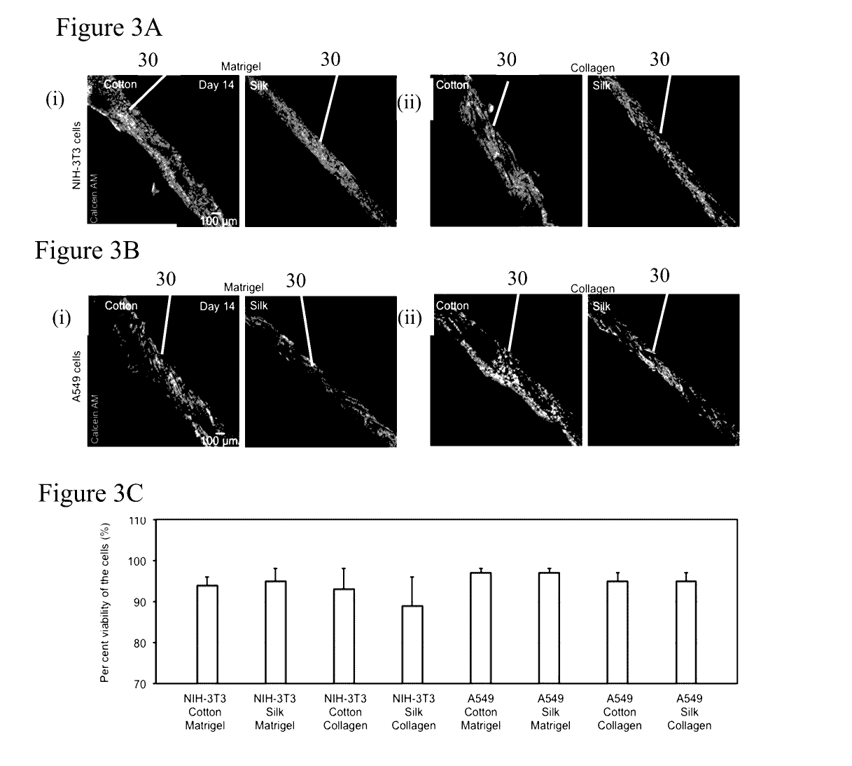

As demonstrated in FIGS. 3A and 3B, the strings did not cause cytotoxic effects on the mammalian cells. The fluorescent confocal microscope images show that cells remained viable throughout the depth of the thread, as indicated by the presence of green fluorescent Calcein (indicated by 30) throughout the string. Quantitative results plotted in FIG. 3C indicated that the viability of cells was greater than 89% within the string scaffolds for all of the cases (n=3). No red stain (indicating dead cells) was observed.

### Formation of Aligned and Organized Cellular Structures in Cells-in-String

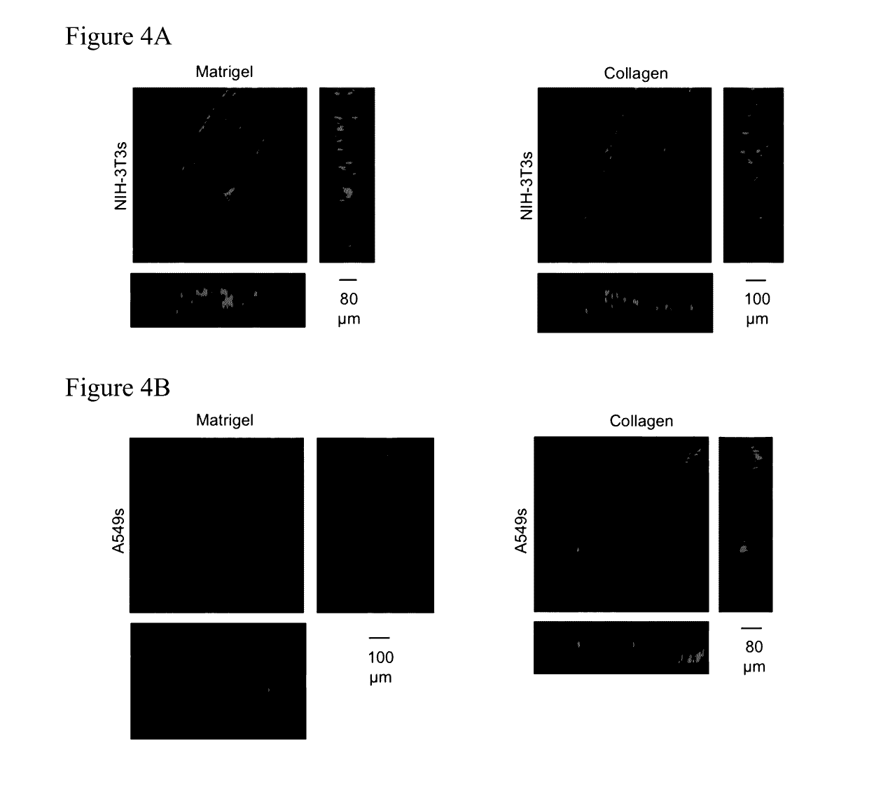

The 3D distribution of the cells in the string scaffolds was investigated in order to understand the organization of the cellular structure within the string scaffold. NIH-3T3 fibroblasts and A549 human lung cancer cells were grown in cotton and silk strings for two weeks. The cytoskeleton (F-actin) and the nuclei of the cells were stained with Texas Red Phalloidin and DAPI, respectively, to demonstrate the morphology within 3D string constructs. The samples were fixed using 4% (v/v) paraformaldehyde. A solution of 0.5% (v/v) Triton X-100 was used to permeabilize the cells. FIGS. 4A and 4B shows fluorescent confocal microscope images of cross-sectional views of the strings and demonstrates the 3D distribution of cells. FIG. 4A shows NIH-3T3 cells in Matrigel (left) or collagen (right) in silk string following 14 days in culture. FIG. 4B shows A549 cancer cells in Matrigel (left) or collagen (right) in silk strings after 14 days in culture.

Due to the smaller diameter and more compact nature of the strands in the silk strings, cells exhibited a closely packed configuration compared to those in the cotton strings. Another parameter that altered the organization of the cells in the string construct was the type of the hydrogel matrix. Matrigel provided a tighter configuration of cells in both cotton and silk strings, possibly due to the physical properties of the hydrogel matrix.

### Metabolic Activity of Cells-in-String

The metabolic activity of the cells in the string scaffolds was quantified using a colorimetric Alamar Blue assay. The cell-laden strings were prepared and cultured as described above, and the cell-loaded string constructs were incubated with the Alamar Blue reagent that was mixed with media in 1:9 proportions for 4 h at 37° C. Alamar Blue was reduced by the metabolically active cells altering the color of the media. The Alamar Blue dye in the media is chemically reduced in the presence of the metabolically active cells, which causes formation of red color. The resulting color was quantified by reading the absorbance at 570 nm and 600 nm by a SpectraMax microplate reader (Molecular Devices, LLC, Sunnyvale, Calif.).

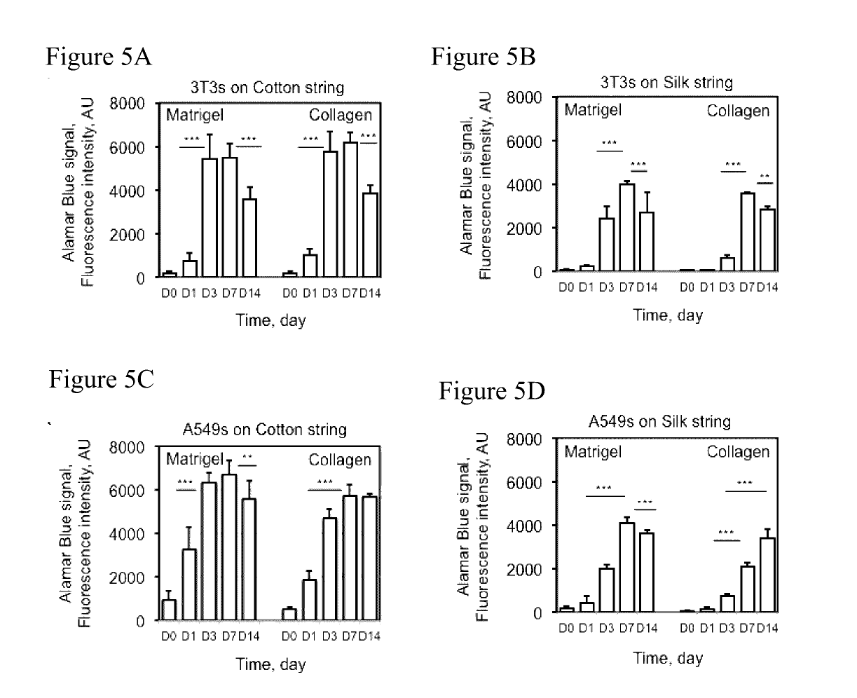

NIH-3T3 fibroblasts had similar metabolic activity in both collagen and Matrigel. As shown in FIGS. 5A and 5B, the metabolic activity of NIH-3T3s increased until day 7 and decreased afterwards on both cotton and silk strings. The cellular activity was, however, different depending on the type of the string, with metabolic activity of the NIH-3T3 cells having lower activity on silk thread. The metabolic activity of the A549 lung cancer cells in Matrigel and Collagen matrices in cotton and silk were similar to that of the NIH-3T3s. As shown in FIGS. 5C and 5D, A549 cells also demonstrated an increasing trend in their metabolic activities until day 7 in both Matrigel and collagen. Further culture of the cells after 7 days resulted in a decrease in their metabolic activities on day 14. The metabolic activity of A549 cancer cells was different depending on which type of string they were cultured in. The metabolic activity of A549s was higher on cotton than that of the silk strings. This result could potentially be due to the differences in the material properties of cotton and silk.

### 3D Patterns of Cells-in-String and Co-Cultures

The cells in string construct allowed for generation of patterns of co-cultured cells in 3D. In addition to patterns of only strings, different patterns of can be formed. The cell-laden string scaffolds were arranged in different patterns and cultured for two days. FIG. 6A demonstrates different patterns used to lay down the strings, shown in the schematic in the upper portion of the figure, and the resulting cell pattern of the cultured cells in the lower portion of the figure. The left hand images shows a single string embedded with H9 lymphocyte cells 66. The center image shows a cross pattern of two strings impregnated with GFP-A549 lung cancer cells 62 and NIT-3T3 fibroblasts 60. The right hand image shows three strings arranged in a triangular pattern formed by T84 colon epithelial cells (64), GFP-A549 lung cancer cells (62), and H9 lymphocytes (66) after culturing two days.

In the experiment shown in FIG. 6B, the cell-laden strings were used as a patterning tool to generate patterns of cells on gel-impregnated Whatman filter paper. The string scaffolds were seeded with cells in a hydrogel matrix and cultured for one or two days day to allow for gelation and integration of the cells. Whatman 114 filter paper was sterilized by autoclaving and impregnated with collagen type I or Matrigel. The gel-impregnated paper solidified for two hours at 37° C. The NIH-3T3 fibroblasts were labelled by cell tracker in blue (string 67 shown in FIG. 6B) and cultured in cotton strings with GFP-A549 human lung cancer cells for one day. The cell-laden strings were then crossed in the pattern shown in FIG. 6B (single string with D1.1 cells 65 (left), crossed strings with NIH-3T3 cells 61 and GFP-A549 cells 63 (center) and single string with H9 cells 67 on GFP-A549 seeded paper 63) and sandwiched between two layers of filter paper impregnated with Matrigel. Following two days of culture, the paper samples were imaged with confocal microscopy, and demonstrated that the cellular patterns on the strings were transferred to the filter paper.

FIG. 6C illustrates a further pattern of cell culturing in which GFP-A549-laden cotton string 68 was cultured on a collagen gel.

### Response of Cells-in-Strings to Toxins/Drugs

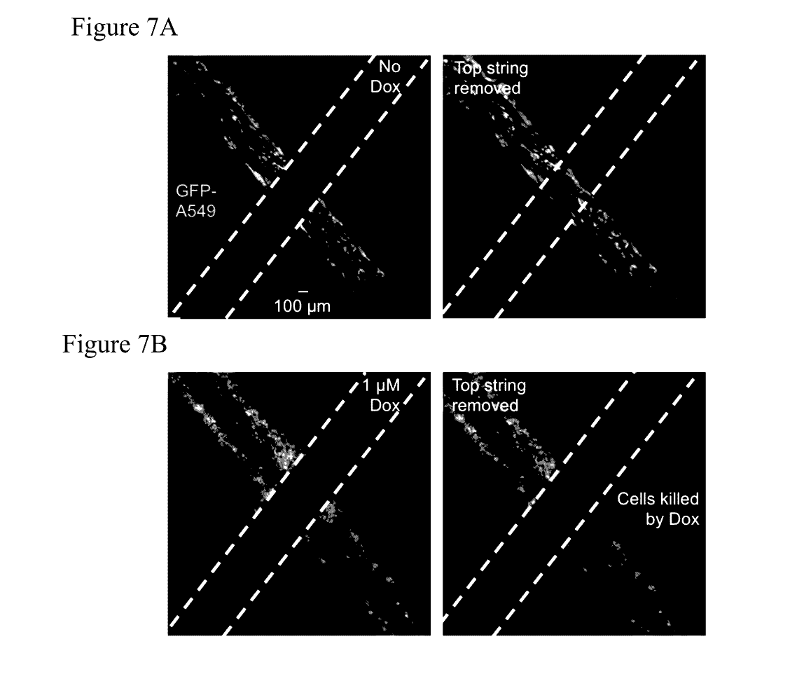

Cells in string were evaluated for the effect of a toxic drug on cancer cells. Green fluorescent protein (GFP) expressing A549 human lung cancer cells were grown in cotton string in Matrigel for two days. In another string, 1 mM Doxorubicin was encapsulated, which is a toxic drug. Doxorubicin was selected due to its potency in cancer treatment. These two strings were then crossed and sandwiched between two layers of filter paper that were impregnated with Matrigel. This assembly was cultured for two days and the cellular patterns that were transferred to the paper were imaged. One sample included a string that did not contain any doxorubicin as a control (FIG. 7A). The top string in the control experiment did not contain doxorubicin and therefore did not cause cell death. A second experiment included the doxorubicin-laden string (FIG. 7B). The confocal microscopy images indicated that the cells in the string died on the region where they contacted the top string containing doxorubicin. The results indicate that the cell in strings construct can easily be adapted for drug screening against various diseases including infectious or non-communicable diseases.

### String is a Suitable Matrix to Culture Bacteria

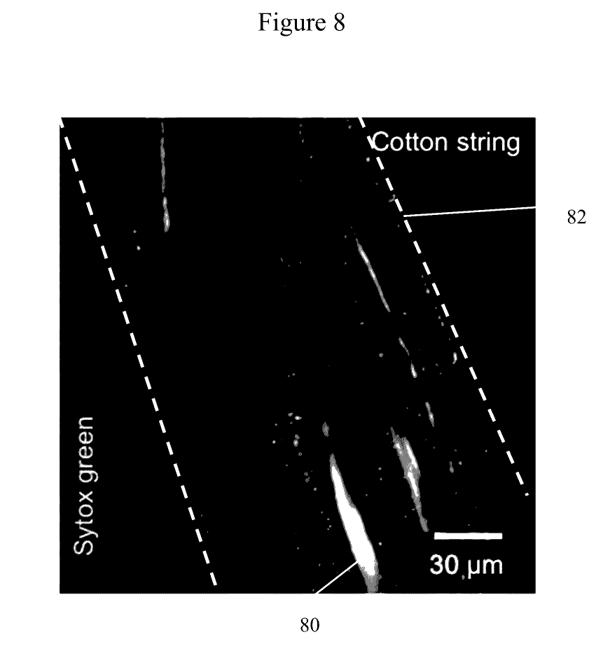

In addition to culturing mammalian cells, strings were also utilized to grow bacteria. E. coli 25922 was grown in cotton string scaffold for three days and a live/dead assay was performed to determine the viability of the bacteria. The live cells were shown in green (80) in the confocal microscopy image (FIG. 8) and show that the living cells populated the full three dimension of the string. Dead cells (Propidium iodide (PI)) were shown in red (82) in the confocal microscopy image (FIG. 8), and showed only a few pinpoint isolated regions of dead cells. The string constructs supported high bacterial cell viability over three days.

Unless otherwise defined, used or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein. For example, if a particular composition is referenced, the composition may be substantially, though not perfectly pure, as practical and imperfect realities may apply; e.g., the potential presence of at least trace impurities (e.g., at less than 1 or 2%) can be understood as being within the scope of the description; likewise, if a particular shape is referenced, the shape is intended to include imperfect variations from ideal shapes, e.g., due to manufacturing tolerances. Percentages or concentrations expressed herein can represent either by weight or by volume.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments. Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. Further still, in this disclosure, when an element is referred to as being “on,” “connected to,” “coupled to,” “in contact with,” etc., another element, it may be directly on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise.

It will be appreciated that while a particular sequence of steps has been shown and described for purposes of explanation, the sequence may be varied in certain respects, or the steps may be combined, while still obtaining the desired configuration. Additionally, modifications to the disclosed embodiment and the invention as claimed are possible and within the scope of this disclosed invention.

What is claimed is:

1. A cell scaffold comprising
a string having a diameter of less than 500 μm;
a hydrogel matrix supported in the string; and
cells seeded into the hydrogel matrix.

2. The cell scaffold of claim 1, wherein the cells are bacterial cells, insect cells, yeast cells, or mammalian cells.

3. The cell scaffold of claim 1, wherein the hydrogel is a temperature sensitive hydrogel.

4. The cell scaffold of claim 1, wherein the hydrogel is an ionotropic hydrogel.

5. The cell scaffold of claim 1, wherein the hydrogel is selected from alginic acid (AA), carboxymethylcellulose (CMC), [symbol] -carrageenan, poly(galacturonic acid) (PG), poly(bis(4-carboxyphenoxy)-phosphazene, guar gum, gellan gum, PuraMatrix hydrogel, poly(ethylene glycol) (PEG), poly(2-hydroxyethyl methacrylate) (PHEMA), poly(N-isopropylacrylamide) (PNIPAAM) gelatin, heparin, agarose, fibrin, pullulan, and dextran and mixtures thereof.

6. The cell scaffold of claim 1, wherein the hydrogel is collagen or Matrigel hydrogel.

7. The cell scaffold of claim 1, wherein the string has a diameter in the range of 50-500 μm.

8. The cell scaffold of claim 1, wherein the string has a diameter in the range of 100-200 μm.

9. A cell sheet structure comprising:
at least one cell scaffold comprising a string having a diameter of less than 500 μm; a hydrogel matrix supported in the string; and cells seeded into the hydrogel matrix; and
a pair of hydrogel impregnated sheets, wherein the at least one cell scaffold is disposed between the pair of hydrogel sheets.

10. The cell sheet structure of claim 9, the structure comprising first and second cell scaffolds.

11. The sheet structure of claim 10, wherein the first cell scaffold comprises cells of a first type and the second cell scaffold comprises cells of a second type.

12. The cell sheet structure of claim 10, wherein the first and second cell scaffold comprise cells of a first cell type.

13. The cell sheet structure of claim 9, wherein the at least one cell scaffold comprises cells of a first cell type; and further comprising a second string comprising an agent of interest, wherein the at least one cell scaffold and the second string are disposed between the pair of hydrogel sheets and in contact with one another.

14. The cell sheet structure of claim 13, wherein the agent of interest is selected from the group consisting of a toxin, drug, serum, growth factor, cytokine, neurotransmitter, chemoattractant, or anti-mitotic agent and combinations thereof.

15. The cell sheet structure of claim 9, where the sheets are paper.

16. A method of culturing cells comprising:
providing a cell scaffold comprising a string having a diameter of less than 500 μm; a hydrogel matrix supported in the string; and cells seeded into the hydrogel matrix; and
incubating the cell scaffold under conditions that promote cell growth.

17. The method of claim 16, further comprising evaluating organization of the cellular structure within the cell scaffold.

18. A method of evaluating cell viability, comprising:
providing a cell scaffold comprising a string having a diameter of less than 500 μm; a hydrogel matrix supported in the string; and cells seeded into the hydrogel matrix;
incubating the cell scaffold; and
qualitatively or quantitatively determine the level of viable cells in the cell scaffold.

19. The method of claim 18, further comprising contacting the cell scaffold with a second string comprising an agent of interest; and
determining the level of viable cells in the cell scaffold in the presence of the agent of interest.

20. The method of claim 19, wherein the agent of interest is selected from the group consisting of a toxin, drug, serum, growth factor, cytokine, neurotransmitter, chemoattractant, or anti-mitotic agent and combinations thereof.

21. A method of evaluating cell metabolic activity, comprising:
providing a cell scaffold comprising a string having a diameter of less than 500 μm; a hydrogel matrix supported in the string; and cells seeded into the hydrogel matrix;
incubating the cell scaffold in the presence of an agent capable of assessing metabolic activity; and
qualitatively or quantitatively determining the level of metabolic activity as a function of the agent capable of assessing metabolic activity.

22. The method of claim 21 further comprising contacting the cell scaffold with a second string comprising an agent of interest; and
assessing the metabolic activity of the cells in the presence of the agent of interest.

23. The method of claim 22, wherein the agent of interest is selected from the group consisting of a toxin, drug, serum, growth factor, cytokine, neurotransmitter, chemoattractant, or anti-mitotic agent and combinations thereof.

24. A method of patterning cells onto surface comprising:
providing a first cell scaffold comprising a string having a diameter of less than 500 μm;
a hydrogel matrix supported in the string; and cells having a first cell type seeded into the hydrogel matrix;
disposing the first cell scaffold between a pair of hydrogel sheets;
incubating the cell scaffold-hydrogel sheet assembly; and
separating the incubated cell scaffold from the pair of hydrogel sheets, wherein the first cell type is patterned on the pair of hydrogel sheets.

25. The method of claim 24, further comprising:
providing a second cell scaffold comprising a string having a diameter of less than 500 μm; a hydrogel matrix supported in the string; and cells having a second cell type seeded into the hydrogel matrix;
disposing the second cell scaffold between a pair of hydrogel sheets;
incubating the cell scaffold-hydrogel sheet assembly comprising the first and second cell scaffolds; and
separating the incubated cell scaffold from the pair of hydrogel sheets, wherein the first cell type and the second cell type are patterned on the pair of hydrogel sheets.
